(19) United States
(12) Patent Application Publication
(10) Pub. No.: US 2026/0324658 A1
(43) Pub. Date: Oct. 22, 2026

(54) AN APPARATUS AND A METHOD FOR FILLETING A FISH

(71) Applicant: MAREL SALMON A/S, Stovring (DK)

(72) Inventor: Casper MILLER SIMONSEN, Stovring (DK)

(21) Appl. No.: 19/491,482

(22) PCT Filed: Jun. 21, 2024
(86) PCT No.: PCT/EP2024/067503

§ 371 Date: Dec. 9, 2025

(30) Foreign Application Priority Data
Jun. 23, 2023 (EP) 23181212.4

(51) Int. Cl. A22C 25/16; A22C 25/08; G01N 33/12

(52) U.S. Cl. CPC A22C 25/16; A22C 25/08; G01N 33/12

(57) ABSTRACT

A method and an apparatus for filleting a fish. The apparatus includes: a conveyor for conveying the fish in a conveying direction with a backbone of the fish following a center plane of the apparatus and with lateral sides of the fish on opposite sides of the center plane; two adjacent knifes arranged on opposite sides of the center plane for cutting away the lateral sides from the backbone; and a shaping structure arranged to engage the lateral sides to obtain a desired shape of the lateral sides when the fish is engaged by the knifes. To increase yield and provide a more consistent way of filleting the fish, the apparatus includes an electronic control structure configured to read a deformability parameter and to control the shaping structure to engage the lateral sides with a pressure depending on the deformability parameter.

### INTRODUCTION

The present invention relates to an apparatus and a method for filleting fish.

### BACKGROUND

Fish processing operations typically include removal of the heads and the fish entrails. Subsequently, the fish is split longitudinally and vertically into two fish fillets and the backbone. Herein, this subsequent splitting is referred to as filleting. This process is described inter alia in EP1526776B1 which is hereby incorporated by reference. Additionally, a trimming process is disclosed inter alia in EP3255996B1 which is hereby incorporated by reference.

The spine part of the fish is the part extending upwardly from the backbone to the upper back of the fish. The lateral sides are on opposite sides of the backbone and include the belly part of the fish extending downward from the backbone to the abdomen or belly. The center plane of the fish is a longitudinally extending vertical plane intersecting through the middle of the backbone in the middle between the two lateral sides and thus the fillets. The fillets are the pieces of meat arising when the fish is split as described above.

Fish filleting is widely automated and different types of machines use either rotating circular blades or band blades. The band blades have flexibility to allow cutting in curved shapes and therefore cutting closer to the backbone and therefore allow a larger amount of the meat to remain on the fillets.

However, not even band blades allow a completely close cut against the bone, e.g., against the ribs which stick out from the backbone, and in practice, there is typically a balance between the desire to avoid cutting into the bones and the desire to avoid meat on the bones since that reduces the yield.

Some machines incorporate force providing structures, e.g., in the form of elongated finger-shaped bars pressing against the outer surface of the fish during the filleting. While this may smoothen out uneven, curved, shapes of the fish and therefore facilitate the filleting process, it does not completely solve the problem of meat being wasted on the bones during the filleting.

Particularly, the pressure of the force providing structures may need adjustment and changing quality and various conditions of the fish may either prevent optimal yield or necessitate frequent adjustment.

As an example, variations in temperature of the fish, different fat content of the fish, varying duration between slaughtering and filleting, different sizes of the fish, and other conditions of the fish may necessitate frequent adjustment and may lead to a reduced yield in filleting processes.

### DESCRIPTION

On the above background it is an object of embodiments of the present disclosure to provide an improved way of filleting fish and to allow for a better yield. It is a further object to provide a fully automated and accurate solution for filleting, and to allow a higher degree of flexibility when it comes to the quality and conditions of the fish.

For these and other objectives, the present disclosure, in a first aspect, provides an apparatus for filleting a fish in which the fish is shaped during the filleting process by a shaping structure which applies a pressure to the fish depending on how compact and shapable the fish appears to be.

### The Apparatus comprises

a conveyor for conveying the fish in a conveying direction with a backbone of the fish following a center plane of the apparatus and with lateral sides of the fish on opposite sides of the center plane;
a cutting structure for cutting away the lateral sides from the backbone; and
a shaping structure arranged to engage the lateral sides to obtain a desired shape of the ribs and lateral sides.

To ensure that the shaping structure provides a consistent and intended impact on the fish, the apparatus comprises an electronic control structure configured to read a deformability parameter which defines an ability of the fish to become deformed and to control the shaping structure such that it engages the lateral sides with a pressure depending on the deformability parameter.

When the impact of the shaping structure on the fish is consistent, it enables a consistent reshaping of the fish, i.e., the desired shape may be obtained more consistently. Accordingly, the filleting knife(s) can be adjusted to follow the meat closer to the bones and the yield may be improved without compromising the quality.

The apparatus may form part of a machine for fish processing, e.g., a machine incorporating trimming of fat, gutting, or other fish processing steps upstream in the conveying direction, or incorporating different processes downstream the conveying direction.

The conveyor may consist of two conveyor belts arranged in parallel to allow the fish to be conveyed in a gap between the two belts. The two belts could be endless belts, and the belts could include different features for increasing the friction between the surface of the fish and the surface of the belt.

The center plane of the apparatus is a virtual plane introduced for describing the functionality of the machine. The center plane may particularly be a vertical center plane. In one embodiment, the apparatus includes a physical center plate along which the fish slides through the apparatus. Alternatively, the apparatus may include a number of saddles moving with the fish through the apparatus.

The cutting structure may include one or more knives, e.g., circular blades, belt blades, or any other kind of knife structure.

The apparatus may comprise a deformability meter structure arranged upstream the shaping structure in the conveying direction and configured to determine the deformability parameter. Each time a fish is filleted, the electronic control structure may receive data from the deformability meter structure and calculate a corresponding force by which the shaping structure should reshape the fish.

The deformability meter structure may function by applying pressure to the fish and by determining a deformation of the fish obtained by the pressure. The ratio between the pressure and the deformation may indicate the deformability of the fish.

The term “deformability” when used herein denotes how easy it is to deform the fish, i.e. how dense the fish is, i.e., it could alternatively be referred to as a density even though it is not necessarily an exact measure of weight per volume unit etc. but a more general indication of denseness and therefore typically a measure of hardness and/or elasticity. Accordingly, the corresponding deformability parameter does not refer to a measure of weight per cubic unit or a shore hardness or a coefficient of elasticity etc., but simply a number defining how easy it is to deform the fish relative to other fish in the row of fish being processed. Accordingly, the term deformability could alternatively be density, the term deformability parameter could be density parameter, the term deformability meter structure could be a density meter structure, and the term deformability sensor could be density sensor.

The deformability parameter of the fish, dF, could be provided by the following equation which herein is one example of a deformability parameter equation:

dF=dimp/dP

where:
dimp is the impact, i.e., the degree of deformation of the fish. This could e.g., be a change in width obtained when changing between different pressures or it could be a penetration depth when penetrating an element into the fish. dimp could be measured e.g., in millimeter (mm). dP is the change in pressure or force against the fish providing the impact. For a specific structure being pressed against the fish, dP could be measured in Newton (N).

I.e., the deformability parameter expresses the impact of the pressure, e.g., the change in the width of the fish when the fish is compressed sideways.

The pressure may be applied by the deformability meter structure by what is referred to herein as a deformability sensor. The deformability sensor could be a structure which is pressed against the fish by any suitable mechanism such as magnetism, e.g., by an electrical motor, or by pneumatic or hydraulic forces. The deformability sensor could have any surface shape towards the fish, e.g., a flat or spherical shape facing the fish.

The deformability meter structure may comprise several deformability sensors arranged along the conveyor, and each deformability sensor may be arranged to apply the pressure at different locations on the fish. This could e.g., be near the head end, near the tail end, at the thickest point on the fish, or elsewhere on the fish.

Particularly, the deformability meter structure may provide an electrical deformability signal, e.g., a digital signal, e.g., communicated by cable or wirelessly to an electronic controller. Based on the electrical deformability signal, the electronic controller controls the shaping structure, e.g., by controlling said actuator structure operatively associated with the internal or external pressure structures. This may include applying different pneumatic pressures to pneumatic actuators to control a pneumatically generated force onto the fish or applying different electrical control signals to an electrical actuator for applying an electrically generated force to the fish.

When viewed in the conveyance direction, the distance between the shaping structure and the knives may be smaller than the distance between the shaping structure and the deformability meter structure. This allows the shaping structure and the deformability meter structure to be completely separated and reduces the risk that the deformability meter structure should be influenced by the pressure applied by the shaping structure.

The deformability meter structure may comprise at least two deformability sensors both configured to measure a position of the fish relative to the center plane and at least one of the deformability sensors could be configured to apply a measuring pressure to the fish while measuring the position of the fish in the machine.

The at least two deformability sensors could be located at a distance from each other in the conveying direction.

A deformability sensor could be a sensor having a contact element by which the surface of the fish is contacted, and optionally compressed. During this process, the ratio between the contact pressure and the deformation of the fish is recorded and converted into the deformability parameter. In one example, the deformability parameter could be a scale from a lowest value pertaining to a low deformability to a highest value pertaining to a high deformability, e.g., a digit from 1 to 10.

The at least two deformability sensors may be configured to apply different measuring pressures to the fish, and/or to apply the measuring pressure in different directions, and/or to apply the measuring pressure at different locations of the fish.

At least one of the at least two deformability sensors could be arranged to apply the measuring pressure at a location of the fish where the distance from the center plane to the lateral sides of the fish is larger than at any other location of the fish. This location may e.g., be where the maximum width of the fish is found.

At least one of the at least two deformability sensors could be configured to provide an additional measure of the fish. In one example, it is constituted or integrated into a sensor which measures a dimension, e.g., the thickness of the fish, or a sensor which registers a position of the fish when it travels in the conveying direction.

The deformability meter structure may e.g., be configured to calculate an average value from different deformability sensors, or it could be configured to calculate a difference between measurements of the at least two deformability sensors. In this case, the actual deformability parameter could be based on said average or said difference, i.e., the shaping structure is controlled based on the average or the difference.

The shaping structure may comprise an external pressure structure configured to apply the pressure on an outer surface of the lateral sides. The pressure may press the lateral sides towards each other or away from each other to obtain the desired shape suitable for the filleting process.

The external pressure structure may include a plurality of pressure elements arranged in a row extending transverse to the conveying direction, e.g., in the shape of a plurality of elongated rods or beams exerting a pressure on the abdomen part in the direction towards the center plane.

Each pressure element may be movable towards the center plane by an actuator structure, e.g., by magnetism, an electrical motor, or under influence of pressure of a medium, e.g., air pressure, or a hydraulic pressure. The actuator structure may be controlled by the deformability parameter such that the actuator structure applies pressure on the fish via the external pressure structure depending on the deformability parameter.

Additionally, the shaping structure may comprise an internal pressure structure configured to apply the pressure on an inner surface of the lateral sides pressing the lateral sides away from each other. The internal shaping structure may comprise internal structures following the inner surface of the lateral sides of the fish during the filleting process. The internal structures may e.g., be plate shaped members, e.g., parallel to the center plane or nearly parallel to the center plane and being movable in a direction away from the center plane to thereby push the lateral sides of the fish away from the center plane.

Each internal structure may be movable by an actuator structure, e.g., by magnetism, an electrical motor, or under the influence of pressure of a medium, e.g., air pressure, or hydraulic pressure. The actuator structure may be controlled by the deformability parameter such that the actuator structure applies pressure on the fish via the internal pressure structure depending on the deformability parameter.

The shaping structure may be configured to provide the desired shape simultaneously with the fish being engaged by knives of the cutting structure.

The cutting structure may comprise two adjacent knives arranged on opposite sides of the center plane for cutting away the lateral sides from the backbone.

The two adjacent knives may be arranged for cutting away the lateral sides from the backbone and potentially from the ribs. The knives could be circular blades, band blades, or generally any kind of blades for cutting fillets. If the knives are constituted by band blades, a squeezing structure may be arranged for urging the bands towards each other on opposite sides of the backbone of the fish. In this way, the bands may curve around the backbone and cut closely to the backbone.

The apparatus may comprise a width measuring structure configured to determine a width signal representing the width of the fish. Herein, the term “width” denotes a dimension perpendicular to the center plane. The width measuring structure may e.g., be optical or mechanical. An example of a mechanical width measuring structure is a hinged structure, e.g., a plate, an arm or similar hinged structure movable up and down when a fish enters with one lateral side on the conveyor and the opposite lateral side facing the hinged structure. In this embodiment, the width measuring structure may constitute one of said two deformability sensors.

Said shaping structure may be configured to engage the lateral sides of the fish with a pressure depending on the determined width of the fish. For this purpose, the width measuring structure may be arranged to communicate the width signal to the electronic control structure which then controls the shaping structure based on the width signal.

The apparatus may comprise a length measuring structure configured to determine a length signal representing the length of the fish in the conveying direction.

The length measuring structure may e.g., be optical or mechanical. An example of a mechanical length measuring structure is a hinged structure, e.g., a plate, an arm, or similar hinged structure movable up and down when a fish enters with one lateral side along the conveyor and the opposite lateral side following the hinged structure. In this embodiment, the length measuring structure may constitute one of said two deformability sensors. Furthermore, the length measuring and width measuring structure may be constituted by the same sensor.

The electronic control structure may be configured to read the length signal and control the shaping structure based thereon such that pressure is applied to the fish depending on the length.

The electronic control structure comprises a data interface configured for communication of the deformability parameter with an external data interface. This may enable receival of the deformability parameter from a previous apparatus in a chain of devices constituting a fish processing system, or it may enable an operator to key-in a value of the deformability parameter, e.g., based on exact knowledge about a batch of fish, or based on experience with a certain type of fish, or based on an experienced manual compression of the fish by hand.

The fish may change the deformability parameter during the period of time following the slaughtering of the fish, particularly as a result of the pre and post rigor states. The electronic controller may be configured to translate a certain duration after slaughtering into a deformability parameter. Such a feature may involve an empirical model included in the controller and configured, e.g., based on a selection of a specific kind of fish from a library and a duration after slaughter, to determine an estimate of the deformability parameter.

In a second aspect, the disclosure provides a method of filleting a fish. According to the method, the fish is conveyed in a row of fish following a conveying direction with a backbone of the fish following a virtual center plane through the apparatus and with lateral sides of the fish being on opposite sides of the center plane of the apparatus.

Lateral sides are urged towards each other or away from each other with a force determined based on an ability of the fish to deform the fish while the lateral sides are released from the backbone and the ribs.

The deformability of the fish may be determined for more than one fish in the row of fish, e.g., for each fish in the row of fish such that the shaping structure is adjusted between each fish being filleted. The deformability parameter may be determined by applying a known pressure and determining a corresponding deformation of the fish, particularly by 9/15 applying two different known pressures and determining the corresponding two different deformations of the fish.

### BRIEF DESCRIPTION OF THE DRAWINGS

FIG. 1 is a perspective view of an apparatus for filleting;

FIG. 2 is a perspective view of a shaping structure with an external pressure structure;

FIG. 3 is a perspective view of a shaping structure with an internal pressure structure;

FIG. 4 is a perspective view of a shaping structure combining internal and external pressure structures and visualizing two band blades;

FIG. 5 illustrates a measure structure which can be used both for measuring a measure of the fish, e.g., length or width, and which may simultaneously form a deformability sensor forming part of the deformability meter structure;

FIG. 6 illustrates a conveyor incorporating a deformability sensor; and

FIG. 7 illustrates a process diagram illustrating a method of filleting a fish.

The drawing figures are not necessarily drawn to scale. Instead, they are drawn to provide a better understanding of the components and are not limited in scope but to provide exemplary illustrations. The figures illustrate exemplary configurations of the apparatus and in no way limit the structures or configurations of the apparatus according to the present disclosure.

### DETAILED DESCRIPTION OF VARIOUS EMBODIMENTS

Embodiments of an apparatus overcome limitations of existing systems by providing an apparatus that advantageously allows for better yield without compromising the quality of the fillets of a fish.

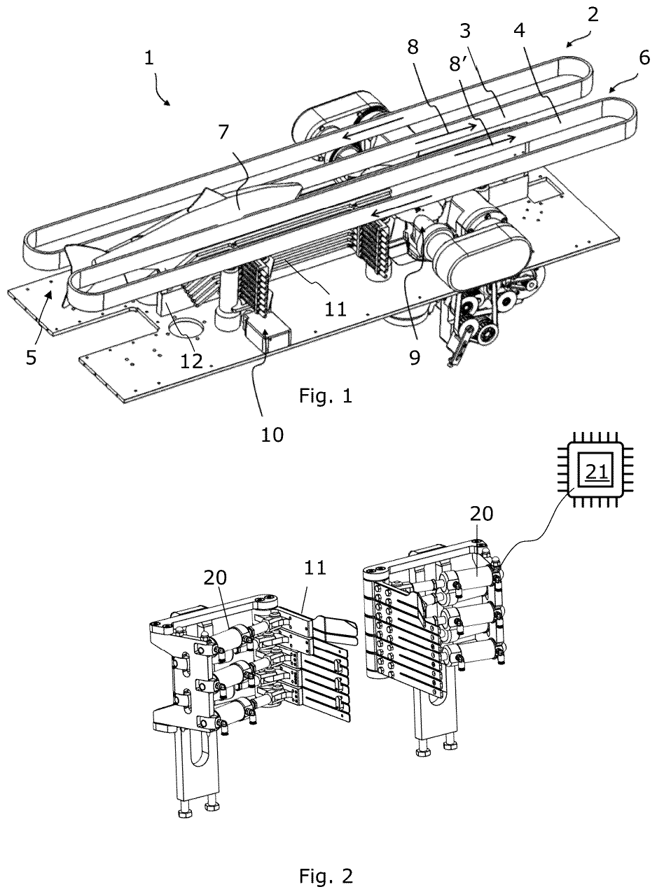

FIG. 1 illustrates an apparatus 1 comprising a conveyor 2 comprising two endless belts 3, 4 defining an entry 5 and an exit 6. Conveyor 2 conveys fish 7 in a conveying direction illustrated by arrows 8, 8′. The two endless belts are arranged side-by-side with adjacent sides of the belts against the fish to convey the fish from the entry to the exit.

In the illustrated apparatus, the orientation of the fish is such that the head side of the de-capitated and gutted fish points forward in the direction of conveying and the belly is downward.

During conveying through the apparatus, the backbone of the fish follows a virtual center plane of the apparatus, and lateral sides of the fish are on opposite sides of the center plane.

The apparatus comprises a cutting structure 9 configured for cutting away the lateral sides from the backbone. The cutting structure may have any structure known in the art for filleting and generally comprises two adjacent knives arranged on opposite sides of the center plane. The cutting structure may e.g., comprise circular blades, band blades, or any other kind of blades suitable for filleting. FIG. 4 illustrates an example of band blades provided for filleting.

The shaping structure 10 comprises an external pressure structure with a plurality of force supplying elongated beams 11. Each elongated beam interacts with the fish by applying pressure to the lateral side of the fish while the fish advances through the apparatus. By this engaging between the elongated beams and the fish, a desired shape of the fish can be obtained prior to, and/or during the filleting process when the fish is engaged by the knives.

The shaping structure 10 further comprises an internal pressure structure with two plate shaped members 12 entering between the lateral sides of the fish belly part and configured to press the sides with the ribs outwards. Again, this is to obtain a desired shape of the fish prior to, or during the filleting process when the fish is engaged by the knives.

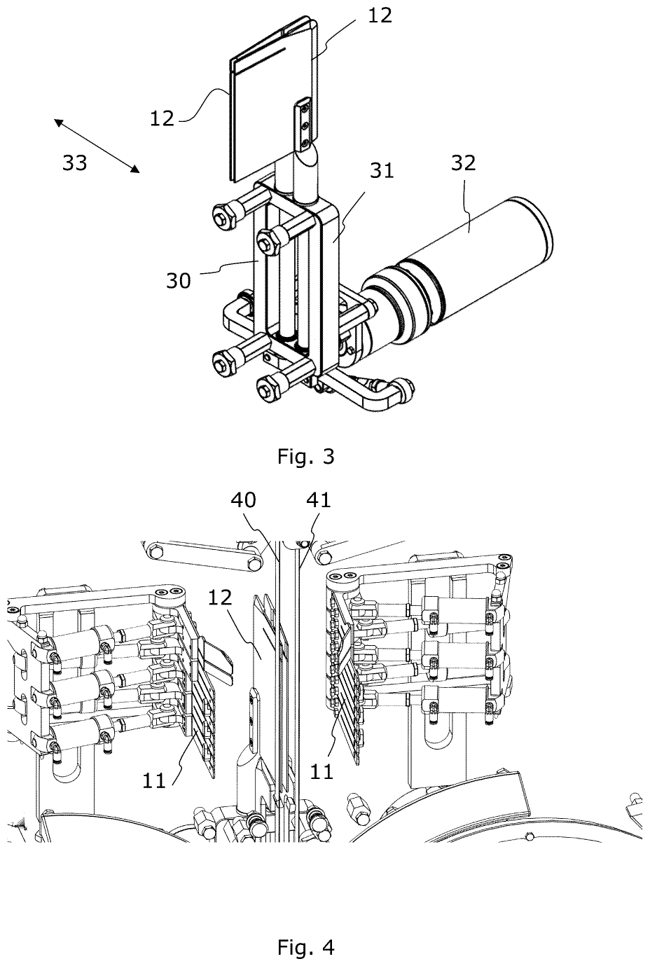

The external pressure structure is illustrated in further detail in FIG. 2 and the internal pressure structure is illustrated in further detail in FIG. 3.

FIG. 2 illustrates details of the internal pressure structure including an actuator structure constituted in this case by pneumatic cylinders 20 operated by air pressure. The actuator structure is controlled by the electronic control structure 21 the pressure is applied to the fish depending on a deformability parameter for the fish.

Electronic control structure 21 may be constituted by a CPU with memory and computer executable code for enabling various functions including reading the deformability parameter and providing control instructions for the actuator structure. Software program instructions and data may be stored on a non-transitory, computer-readable storage medium, and when the instructions are executed by the CPU these functions are carried out.

FIG. 3 illustrates the internal pressure structure configured to apply the pressure on an inner surface of the lateral sides pressing the lateral sides away from each other. The internal pressure structure comprises two plate shaped members 12 held by actuator arms 30, 31 being movable by the actuator structure 32 such that they can move towards each other or away from each other in the direction indicated by the arrow 33. The actuator structure is controlled by the electronic control structure 21 the pressure is applied to the fish depending on a deformability parameter for the fish.

FIG. 4 illustrates a shaping structure comprising both the internal and the external pressure structures 11, 12. The shaping structure is illustrated in combination with a cutting structure with two adjacent band blades 40, 41. The distance, in the conveying direction between the shaping structure and the cutting structure is short such that the desired shape obtained by controlling the shaping structure is maintained while the fish is cut by the band blades. The distance may particularly be less than the length of the fish, e.g., less than half the length of the fish.

The apparatus may comprise a deformability meter structure arranged upstream the shaping structure such that the deformability can be determined for each fish being processed.

The deformability meter structure may function by applying pressure to the fish and by determining a deformation of the fish obtained by the pressure. The ratio between the pressure and the deformation may indicate the deformability of the fish.

The deformability meter structure may comprise several deformability sensors arranged one after the other in the conveying direction. The deformability sensors may be arranged to apply different pressures to the fish and to record a corresponding position of the surface of the fish where the pressure is applied.

The first sensor may e.g., register a width of the fish with a first pressure, and the second sensor may register a width of the fish with a second pressure. The deformability parameter of the fish, dF, could be provided by inserting dimp and dP into the deformability parameter equation dF=dimp/dP.

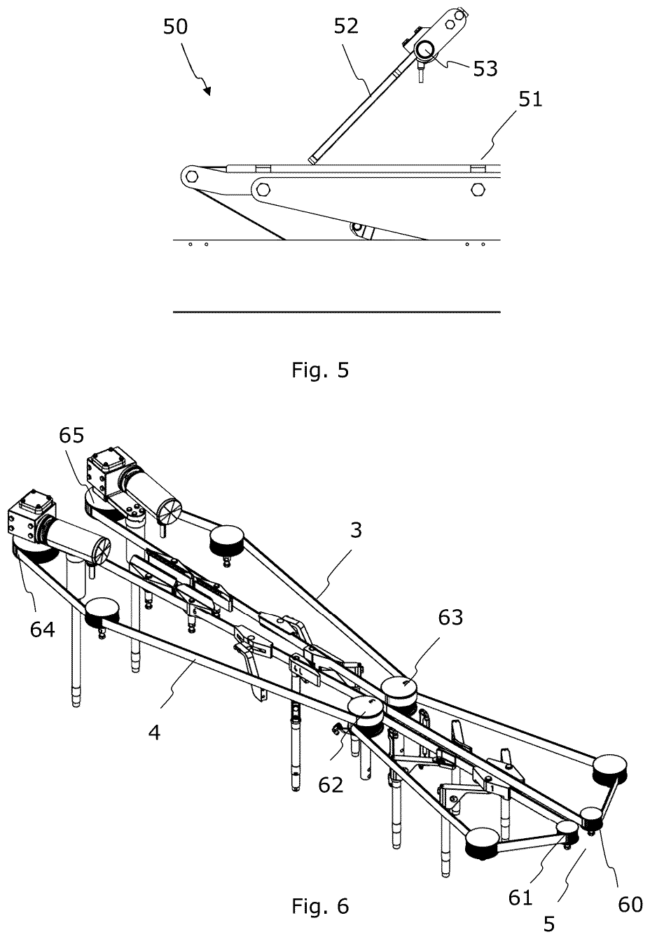

When using several deformability sensors, each sensor may apply a pressure different from the pressure of the other sensors. In one example, a first measuring station comprises a device for measuring the width of the fist. This device may not apply a pressure, or at least only apply a very small and irrelevant pressure which essentially provides no deformation of the fish. This measuring station could be considered as one of the deformability sensors since it provides a width of the fish when no force is applied. Such a station is illustrated in FIG. 5.

A subsequent measuring station may comprise an element being pressed sideways against the fish, and the width may simultaneously be determined. Such a station is illustrated in FIG. 6.

FIG. 5 illustrates a measuring structure 50 configured to determine the width of the fish. The measuring structure comprises a conveyor 51 on which the fish is conveyed in a sideways orientation in which one lateral side faces the conveyor and the other lateral side faces the hinged plate element 52 under which the fish is conveyed. The fish lifts the hinged plate element while the plate element pivots about a pivot point 53. The angular displacement of the hinged plate determines the width of the fish. At the same time, the hinged element exerts almost no pressure on the fish or only a pressure which is inessential to the width of the fish. By registering the ratio between this applied pressure, i.e., almost no pressure, and the corresponding deformation of the fish, the measuring structure provides a measure of a dimension of the fish when essentially no pressure is applied to the fish.

The measuring structure illustrated in FIG. 5 may also, with the same structure, be configured for determining the length of the fish. In this configuration, the length is determined by the speed of the conveyor and by the angular displacement of the plate element. When the fish enters under the plate element, the plate swings upwardly, until the fish has passed through the measuring structure. Accordingly, the length of the fish can be determined based on the speed of the conveyor and the duration from the displacement of the plate element starts until the plate element is back in its lowermost position. At the same time, the hinged element exerts a certain pressure on the fish, i.e., essentially no pressure, and by registering the ratio between this applied pressure and the corresponding deformation of the fish, the measuring structure may be used both for length measurement and as a deformability sensor forming part of the deformability meter structure and providing a first data set of pressure and a corresponding dimension of the fish.

FIG. 6 illustrates an embodiment of conveyor 2 comprising two endless belts 3, 4 defining an entry 5 and an exit 6. The two endless belts are arranged side-by-side and define a gap in which the fish is located.

The illustrated conveyor incorporates a deformability meter structure capable of providing a measure for the deformability of the fish.

A second measuring station is defined at entry 5 of the conveyor. In this position, two rotating belt rollers 60, 61 are moveable towards and away from each other. The movability is controlled via a step motor which can apply a specific force and determine a specific position of the belt rollers. The belt rollers can thereby compress the fish by applying a specific force to the fish, and the position of the rollers can be identified by the step motor. Accordingly, the second deformability sensor provides a second data set of pressure and a corresponding dimension of the fish.

Since the apparatus, in the previous, first deformability sensor, determines a first data set of pressure and a corresponding dimension of the fish, a ratio between the force used for deforming the fish and the deformation of the fish can be calculated.

The belt rollers 60, 61 and the corresponding sensor motor by which they are moved towards each other thereby constitute a deformability sensor.

In the mentioned example, the measuring structure 50 and the two rollers 60, 61 and the corresponding stepper motor communicate with the electronic control structure 21, and the electronic control structure can deduct the deformability and use it for controlling the shaping structure to obtain a desired shape of the fish during the filleting process.

The deformability meter structure may comprise several such deformability sensors for applying the pressure to the fish. FIG. 6 illustrates three sets of belt rollers having the capability of determining deformability, namely the roller set 60/61, the roller set 62/63, and the roller set 64/65, i.e., in this case the apparatus comprises three deformability sensors. At least two of these deformability sensors, and potentially all three deformability sensors may be configured to apply different measuring pressures to the fish. An average of the measurements, or a difference between measurements of the different deformability sensors may be used as a measure for the deformability parameter.

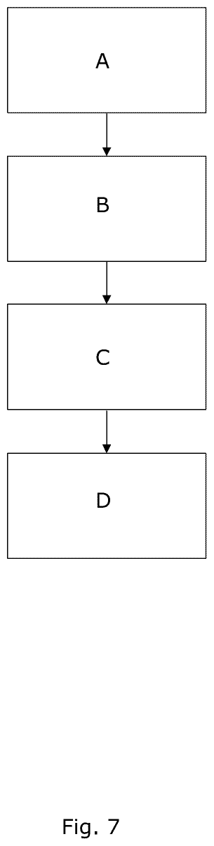

FIG. 7 illustrates a process diagram illustrating a method of filleting a fish. In this diagram, the first process, A is a step of receiving a plurality of fish on a conveyer configured to convey the fish in a row of fish. Step B is a sensing step in which a deformability meter structure determines the deformability parameter by gently compressing the fish while tracking the pressure against the surface of the fish. Step C is a step of determining a control signal for a shaping structure. This step includes a calculation of a force which should be applied onto a surface of the fish by the shaping structure. Finally, step D is a step of urging lateral sides towards each other and/or away from each other with a force determined based on the deformability parameter and thereby an ability of the fish to deform. Step D is executed while the lateral sides are released from the backbone and the ribs. The result of step D is a filleted fish, i.e. two fillets released from the backbone and the ribs.

### EXAMPLES

In one example, each fish is analyzed by the deformability meter structure, and each time a fish is filleted, the electronic control structure receives data from the deformability meter structure and calculates a corresponding force by which the shaping structure should reshape the fish. In this example, the fish may pass e.g., two or three deformability sensors on its way towards the cutting structure for filleting. Each deformability sensor applies pressure to the fish and determines a corresponding impression depth into the fish.

In one embodiment, two deformability sensors arranged one after the other in the direction of conveying and function as follows. The first sensor registers the width of the fish with a first pressure being essentially zero, and the second sensor registers a width of the fish with a second pressure.

As an example, the first deformability sensor may determine the width of the fish to be 80 mm with zero pressure, and the second deformability sensor may determine the width of the fish to be 50 mm with a pressure of 1 N.

The deformability parameter of the fish, dF, could be provided by the deformability parameter equation:

Df=dimp/dp=(80−50)mm/1n=30 mm/n

I.e., the deformability parameter expresses the impact of the pressure on the width of the fish. Alternatively, the deformability parameter may express an impact of a pressure anywhere on the fish and a correspondingly obtained depression into the fish.

In one embodiment, an average of the readings of the two or three sensors is used in a transfer function which as a result provides e.g., a pneumatic pressure for an array of pneumatic actuators operating the internal and external pressure structures of the apparatus.

In another embodiment, a difference between the readings of the two or three sensors is used in a transfer function which as a result provides e.g., a pneumatic pressure for an array of pneumatic actuators operating the internal and external pressure structures of the apparatus.

In another example, one fish is analyzed each time a new batch of fish is received.

When the fish is analyzed by the deformability meter structure, the electronic control structure receives data from the deformability meter structure and calculates a corresponding force by which the shaping structure should reshape all fish from this batch of fish.

1.-27. (canceled)

28. An apparatus for filleting a fish, the apparatus comprising:
a conveyor for conveying the fish in a conveying direction with a backbone of the fish following a center plane of the apparatus and with lateral sides of the fish on opposite sides of the center plane;
a cutting structure configured for filleting the fish;
a shaping structure arranged to engage the lateral sides to obtain a desired shape of the lateral sides;
an electronic control structure configured to read a deformability parameter representing an ability of the fish to deform, and to control the shaping structure to engage the lateral sides with a pressure depending on the deformability parameter during filleting of the fish.

29. The apparatus according to claim 28, comprising a deformability meter structure arranged upstream the shaping structure and configured to determine the deformability parameter.

30. The apparatus according to claim 29, wherein the deformability meter structure is configured to apply a pressure to the fish and to determine a deformation of the fish obtained by the pressure.

31. The apparatus according to claim 30, wherein the deformability meter structure is configured to determine a ratio between the pressure and the deformation.

32. The apparatus according to claim 29, wherein the deformability meter structure comprises at least two deformability sensors for applying the pressure to the fish.

33. The apparatus according to claim 32, wherein the at least two deformability sensors are displaced relative to each other along the conveyor.

34. The apparatus according to claim 32, wherein the at least two deformability sensors configured to apply different measuring pressures to the fish.

35. The apparatus according to claim 32, wherein at least one of the at least two deformability sensors is arranged to apply the measuring pressure at a location of the fish where the distance from the center plane to the lateral sides of the fish is larger than at any other location of the fish.

36. The apparatus according to claim 32, wherein the deformability meter structure is configured to calculate a difference between measurements of the at least two deformability sensors, and
wherein the deformability parameter is based on the difference.

37. The apparatus according to claim 28, wherein the shaping structure comprises an external pressure structure configured to apply the pressure on an outer surface of the lateral sides pressing the lateral sides towards each other.

38. The apparatus according to claim 37, wherein the external pressure structure comprises a plurality of pressure elements arranged in a row extending transverse to the conveying direction.

39. The apparatus according to claim 28, wherein the shaping structure comprises an internal pressure structure configured to apply the pressure on an inner surface of the lateral sides pressing the lateral sides away from each other.

40. The apparatus according to claim 38, comprising an actuator structure arranged to move at least one of the external pressure structure or internal pressure structure, and
wherein the actuator structure is controlled based on the deformability parameter.

41. The apparatus according to claim 28, wherein the shaping structure is configured to provide the desired shape simultaneously with the fish being engaged by knives of the cutting structure

42. The apparatus according to claim 28, wherein the cutting structure comprises two adjacent knives arranged on opposite sides of the center plane for cutting away the lateral sides from the backbone.

43. The apparatus according to claim 28, comprising a width measuring structure configured to determine a width signal representing a width of the fish,
wherein the width of the fish is a dimension perpendicular to the center plane, and
wherein the electronic control structure is configured to read the width signal and control the shaping structure based thereon.

44. The apparatus according to claim 28, comprising a length measuring structure configured to determine a length signal representing the length of the fish in the conveying direction, and
wherein the electronic control structure is configured to read the length signal and control the shaping structure based thereon.

45. A method of filleting a fish wherein a plurality of fish is conveyed in a row of fish following a conveying direction with a backbone of the fish following a virtual center plane of the apparatus and with lateral sides of the fish being on opposite sides of the center plane; and
wherein the lateral sides are urged towards each other and/or away from each other with a force determined based on an ability of the fish to deform while the lateral sides are released from the backbone and the ribs.

46. The method according to claim 45, wherein deformability of the fish is determined for more than one fish in the row of fish.

47. The method according to claim 46, wherein deformability of the fish is determined for each fish in the row of fish.
